(19) United States
(12) Patent Application Publication
(10) Pub. No.: US 2024/0093307 A1
(43) Pub. Date: Mar. 21, 2024

(54) SERUM EXOSOMAL SF3B4 MARKER COMPOSITION FOR DIAGNOSING EARLY STAGE HEPATOCELLULAR CARCINOMA FOR NONINVASIVE IN VITRO DIAGNOSIS

(71) Applicant: AJOU UNIVERSITY INDUSTRY-ACADEMIC COOPERATION FOUNDATION, Suwon-si (KR)

(72) Inventors: Soon Sun KIM, Suwon-si (KR); Jung Woo EUN, Suwon-si (KR); Jae Youn CHEONG, Suwon-si (KR); Hyo Jung CHO, Suwon-si (KR); Ho Chul KANG, Suwon-si (KR); JuA SON, Seongnam-si (KR)

(73) Assignee: AJOU UNIVERSITY INDUSTRY-ACADEMIC COOPERATION FOUNDATION, Suwon-si (KR)

(21) Appl. No.: 18/322,850

(22) Filed: May 24, 2023

(30) Foreign Application Priority Data
Sep. 19, 2022 (KR) 10-2022-0118138

(51) Int. Cl. C12Q 1/6886

(52) U.S. Cl. CPC C12Q 1/6886; C12Q 2600/158

(57) ABSTRACT

The present disclosure relates to a serum exosomal SF3B4 marker composition for diagnosing early stage hepatocellular carcinoma for noninvasive in vitro diagnosis, wherein, by analyzing an expression level of SF3B4 in plasma proteins as well as expression of SF3B4 in exosomes present in sera of hepatocellular carcinoma patients, the possibility as the most suitable liquid biopsy marker was identified, and a new noninvasive biomarker with high diagnostic accuracy in early stage hepatocellular carcinoma for which no reliable biomarker exists at this present was discovered. As such, by using exosomes to identify cancer cell genomes and proteomes without biopsy, the exosomes are highly applicable in the future as a diagnostic biomarker that is useful in a disease group with a high risk in the biopsy such as hepatocellular carcinoma.

### CROSS-REFERENCE TO RELATED APPLICATION

This application claims the benefit of Korean Patent Application No. 10-2022-0118138 filed on Sep. 19, 2022, in the Korean Intellectual Property Office, the entire disclosure of which is incorporated herein by reference for all purposes.

### INCORPORATION-BY-REFERENCE OF MATERIAL SUBMITTED BY U.S.P.T.O. EFS-WEB

This application contains a Sequence Listing, which is being submitted in computer readable form via the United States Patent and Trademark Office Patent Center and which is hereby incorporated by reference in its entirety for all purposes. The XML file submitted herewith, which is named as “NewApp_J251960023_SequenceListing” and is created on May 24, 2023, contains a 4,533 bytes file.

### BACKGROUND

### 1. Field of the Invention

The present disclosure relates to a serum exosomal SF3B4 marker composition for diagnosing early stage hepatocellular carcinoma for noninvasive in vitro diagnosis.

### 2. Description of the Related Art

Hepatocellular carcinoma is one of the most common carcinomas among Koreans, and according to data of National Cancer Registration and Statistics and Statistics Office in 2018, hepatocellular carcinoma is a malignant tumor with a poor prognosis, ranking 6^{th} in incidence and 2^{nd} in mortality. Due to underlying cirrhosis, most hepatocellular carcinoma patients should bear a considerably high risk of bleeding during biopsy, requiring the development of liquid biopsy technology based on blood to diagnose hepatocellular carcinoma at an early stage and acquire genetic information. Unlike biopsy that a significant risk should be taken, the liquid biopsy is noninvasive and simple since diagnosis is carried out with blood. Unlike biopsy, blood may be drawn multiple times during the treatment, which is suitable for predicting treatment responses during the treatment or residual cancer.

An exosome, a small nano-vesicle which is 30-150 nm in size and produced/secreted from most nucleated cells including cancer cells, is a leading substance in a liquid biopsy technology, is detected in various body fluids such as blood, urine, ascites, and saliva, and contains specific genetic information of parent cells in exosomal cargo to deliver to a target cell. Recently, the exosome has been drawing attention as a key substance for cell-cell communication, and cancer cell-derived exosomes have been pointed out as a key substance for penetration and metastasis of tumor. Cancer cell-derived exosomes transmit genetic information of mother cells to other target cells in the tumor microenvironment to make normal cells malignant, predicting that they play a role of avatars of cancer cells, which are mother cells.

On the other hand, in the case of alpha-fetoprotein (AFP) which has been previously applied as a diagnostic marker for hepatocellular carcinoma, the sensitivity thereof in the hepatocellular carcinoma diagnosis is only about 60%, and so far, there is no reliable serum biomarker for hepatocellular carcinoma, such that it is urgent to discover next-generation hepatocellular carcinoma serum biomarkers for early diagnosis, prognosis, and prediction of treatment response.

### PRIOR ART DOCUMENT

### Patent Document

Korean Patent No. 10-2025005 (Registered on Sep. 18, 2019)

### SUMMARY

### Problem to be Solved by the Invention

An object of the present disclosure is to provide a biomarker composition for diagnosing hepatocellular carcinoma, including serum exosomal SF3B4 as an active ingredient.

In addition, another object of the present disclosure is to provide a composition for diagnosing hepatocellular carcinoma including a preparation capable of measuring an expression level of serum exosomal SF3B4 as an active ingredient, and a kit for diagnosing hepatocellular carcinoma including the same.

In addition, another object of the present disclosure is to provide a method of providing information necessary for diagnosing hepatocellular carcinoma, including measuring an expression level of serum exosomal SF3B4.

In addition, another object of the present disclosure is to provide a method of treating hepatocellular carcinoma, including administering an expression or activity inhibitor of serum exosomal SF3B4 to a hepatocellular carcinoma patient.

### Means for Solving the Problem

In order to achieve the above objects, the present disclosure provides a biomarker composition for diagnosing hepatocellular carcinoma, including serum exosomal SF3B4 as an active ingredient.

In addition, the present disclosure provides a composition for diagnosing hepatocellular carcinoma, including a preparation capable of measuring an expression level of serum exosomal SF3B4 as an active ingredient.

In addition, the present disclosure provides a kit for diagnosing hepatocellular carcinoma, including the composition.

In addition, the present disclosure provides a method of providing information necessary for diagnosing hepatocellular carcinoma, including (1) measuring an expression level of SF3B4 from a blood-derived serum exosome sample isolated from a patient; (2) comparing the measured expression level of SF3B4 with a control sample; and (3) determining as hepatocellular carcinoma when the measured expression level of SF3B4 is higher than the control sample.

In addition, the present disclosure provides a method of treating hepatocellular carcinoma, including administering an expression or activity inhibitor of serum exosomal SF3B4 to a hepatocellular carcinoma patient.

### Effects of the Invention

The present disclosure relates to a serum exosomal SF3B4 marker composition for diagnosing early stage hepatocellular carcinoma for noninvasive in vitro diagnosis, wherein, by analyzing an expression level of SF3B4 in plasma proteins as well as expression of SF3B4 in exosomes present in sera of hepatocellular carcinoma patients, the possibility as the most suitable liquid biopsy marker was identified, and a new noninvasive biomarker with high diagnostic accuracy in early stage hepatocellular carcinoma for which no reliable biomarker exists at this present was discovered. As such, by using exosomes to identify cancer cell genomes and proteomes without biopsy, the exosomes are highly applicable in the future as a diagnostic biomarker that is useful in a disease group with a high risk in the biopsy such as hepatocellular carcinoma.

### BRIEF DESCRIPTION OF THE DRAWINGS

FIG. 1 shows a process of exosome isolation and RNA extraction.

FIG. 2 shows results of exosome isolation for identification of specificity of SF3B4 as a serum exosomal diagnostic marker for hepatocellular carcinoma.

FIG. 3 shows results of evaluation on diagnostic performance of SF3B4 as a serum exosomal diagnostic marker for hepatocellular carcinoma.

FIG. 4 shows a result of evaluation on diagnostic performance of SF3B4 in a hepatocellular carcinoma cohort.

FIG. 5 shows results of analysis through measurement of AUC by dividing mUICC stages to determine whether early stage hepatocellular carcinoma is detected and comparison with AFP, which is a conventional serum marker for hepatocellular carcinoma.

FIG. 6 shows results of evaluating diagnostic performance by combining AFP which is a conventional blood marker and serum exosomal SF3B4.

FIG. 7 shows results of evaluating positivity rates of AFP and exosomal SF3B4 in each disease group and positivity rates of exosomal SF3B4 depending on the positivity of AFP in hepatocellular carcinoma patients.

FIG. 8 shows results of hepatocellular carcinoma diagnostic performance of ELISA and autoAb SF3B4 other than exosomal SF3B4.

### DETAILED DESCRIPTION

The present disclosure provides a biomarker composition for diagnosing hepatocellular carcinoma, including serum exosomal SF3B4 as an active ingredient.

“SF3B4” of the present disclosure may be NCBI accession no. NM_005850.5, but is not limited thereto.

The term “diagnosis” as used herein refers to determining of susceptibility of a subject to a particular disease or disorder, determining of whether the subject currently has a particular disease or disorder, determining of the prognosis in the subject with a particular disease or disorder, or therametrics (e.g., monitoring of a condition of the subject to provide information on therapeutic efficacy).

In addition, the present disclosure provides a composition for diagnosing hepatocellular carcinoma, including a preparation capable of measuring an expression level of serum exosomal SF3B4 as an active ingredient.

Specifically, the preparation capable of measuring the expression level of the SF3B4 may be a primer or probe that specifically binds to the SF3B4, but is not limited thereto.

Preferably, the hepatocellular carcinoma may be early stage hepatocellular carcinoma, but is not limited thereto.

In addition, the present disclosure provides a kit for diagnosing hepatocellular carcinoma, including the composition.

The term “primer” as used herein refers to a short nucleic acid sequence having a short free 3′ hydroxyl group that may form a base pair with a complementary template and act as a starting point for replication of template strands. The primer may initiate DNA synthesis in the presence of a reagent (i.e., DNA polymerase or reverse transcriptase) and four different nucleoside triphosphates for polymerization under a condition with an appropriate buffer solution and temperature. PCR conditions and the length of sense and antisense primers may be appropriately selected according to techniques known in the art.

The term “probe” as used herein refers to a nucleic acid fragment such as RNA or DNA corresponding to several to hundreds bases that may specifically form a binding other than mRNA and is labeled also, so that the presence of a specific mRNA and an amount of expression may be detected. The probe may be prepared in a form of an oligonucleotide probe, a single strand DNA probe, a double strand DNA probe, and an RNA probe. The selection of an appropriate probe and hybridization conditions may be appropriately selected according to techniques known in the art.

In addition, the present disclosure provides a method of providing information necessary for diagnosing hepatocellular carcinoma, including (1) measuring an expression level of SF3B4 from a blood-derived serum exosome sample isolated from a patient; (2) comparing the measured expression level of SF3B4 with a control sample; and (3) determining as hepatocellular carcinoma when the measured expression level of SF3B4 is higher than the control sample.

Preferably, the operation (3) is performed by a cut-off value which may have a Ct value (threshold cycle) of 0.85 for the SF3B4, but is not limited thereto.

Preferably, the hepatocellular carcinoma may be early stage hepatocellular carcinoma, but is not limited thereto.

More specifically, a method for measuring the expression level of SF3B4, RT-PCR may use competitive RT-PCR, real-time RT-PCR, RNase protection assay (RPA), Northern blotting, DNA chip, Western blot, enzyme linked immunosorbent assay (ELISA), radioimmunoassay (RIA), radioimmunodiffusion, Ouchterlony immunodiffusion, rocket immunoelectrophoresis, immunohistostaining, immunoprecipitation assay, complement fixation assay, FACS, or protein chip, but is not limited thereto.

In addition, the present disclosure provides a method of treating hepatocellular carcinoma, including administering an expression or activity inhibitor of serum exosomal SF3B4 to a hepatocellular carcinoma patient.

More specifically, the SF3B4 expression inhibitor may be antisense nucleotides, small interfering RNAs (siRNAs), or short hairpin RNAs (shRNAs) that complementarily bind to mRNA of an SF3B4 gene, and the SF3B4 activity inhibitor may be compounds, peptides, peptide mimetics, aptamers, antibodies, or natural products that specifically bind to an SF3B4 protein, but are not limited thereto.

Hereinafter, the present disclosure will be described in detail according to an example embodiment that does not limit the present disclosure. The following example embodiments of the present disclosure are only intended to embody the present disclosure and do not restrict or limit the scope of the present disclosure. Therefore, what may be easily inferred by an expert in the art to which the present disclosure pertains from the detailed description and embodiments of the present disclosure is interpreted as falling within the scope of the present disclosure.

### Experimental Example

The following experimental examples are intended to provide experimental examples commonly applied to each example embodiment according to the present disclosure.

1. Isolation and Verification of Exosomes

(1) Exosome isolation and RNA extraction from hepatocellular carcinoma cells, normal hepatocyte culture media, and patient sera

10 mL of culture media of hepatocellular carcinoma cells and normal hepatocytes as well as 300 ul of sera of normal patients and hepatitis, cirrhosis and hepatocellular carcinoma patients were used, and extraction was performed using Exosome RNA isolation kit (SeraMir™ Exosome RNA Amplification (Cat #RA806A-1)).

Isolation of exosomes from culture media of hepatocellular carcinoma cells and normal hepatocytes: cells grown in 150 mm dish were washed twice with PBS and then added with 15 mL of serum free media, followed by culture for 72 hours. 10 mL of the culture medium was contained in a 15 mL conical tube and centrifuged at 3000 g for 15 minutes to remove cell debris, and then only the supernatant was transferred to a new 15 mL conical tube, vortexed after adding 2 mL of ExoQuick™, stored at 4° C. for a day, and centrifuged at 1500 g for 30 minutes, followed by RNA extraction from the produced pellet.

Isolation of exosomes from sera of normal, hepatitis, cirrhosis, and hepatocellular carcinoma patients: 72 ul of ExoQuick™ was added to 300 ul of patient sera, and the mixture was vortexed, stored at 4° C. for a day, and centrifuged at 13,000 rpm and 4° C. for 2 minutes, followed by RNA extraction from the produced pellet.

After adding 350 ul of lysis buffer to the pellet, pipetting was performed for release, followed by incubation at RT for 5 minutes. After adding 200 ul of EtOH, the mixture was vortexed for 10 seconds.

After preparing a spin column and a collection tube and transferring the entire sample to the column, centrifugation was performed at room temperature and 13,000 rpm for 1 minute, a solution transferred to the collection tube was removed, 400 ul of wash buffer was added, centrifugation was performed at room temperature and 13,000 rpm for 1 minute, a solution transferred to the collection tube was removed, and, after repetitive operations, centrifugation was performed at 13,000 rpm for 2 minutes, followed by a final drying process.

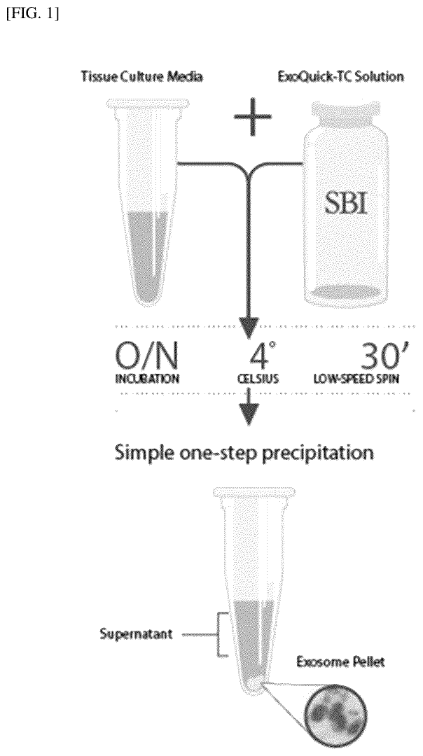

After combining the spin column with an e-tube instead of the collection tube, 30 ul of elution buffer was added, centrifugation was performed at 2,000 rpm for 2 minutes, and final centrifugation was repeated twice at 13,000 rpm for 1 minute. The concentration of the finally obtained solution was measured using Nano Drop (FIG. 1).

(2) Extraction of RNA in Buffy Coat and Sera of Patients

Blood drawn in an EDTA-containing tube or serum-separated tube was collected, and then centrifugation was performed at 2,000 g and 4° C. for 5 minutes to separate buffy coat and sera.

300 ul of buffy coat and sera of normal and hepatocellular carcinoma patients was used, and RNA was extracted using TRIzol™ reagent (Invitrogen).

2. Collection of Blood and Clinical Information of Liver Disease Cohorts for Verification

After passing IRB at Ajou University Hospital, the study was conducted by receiving sera and clinical information of 159 liver disease cohorts (26 normal, 26 with chronic hepatitis, 32 with cirrhosis, and 75 with hepatocellular carcinoma) through Korea Biobank.

3. Measurement of an Expression Level of SF3B4 in Sera and Serum Exosomes of Patients in the Liver Disease Cohort Via qRT-PCR Analysis

300 ul of buffy coat and patient sera was used to measure SF3B4 expression, and cDNA synthesis was performed using TRIzol reagent (Cat #15596018)(Ivitrogen), Exosome RNA isolation kit (SeraMir™ Exosome RNA Amplification (Cat #RA806A-1))(SBI, System Biosciences), PrimeScript™ RT Master Mix (Perfect Real Time)(Cat #RR036A)(TaKaRa), and miScript RT II kit (Cat #218161)(QIAGEN).

Buffy coat RNA or serum RNA and RNA Free Water (RFW) were combined to make the volume of 8 ul, and 2 ul of 5× PrimeScript RT Master Mix was added to make the total volume of 10 ul to carry out cDNA synthesis.

Serum exosomal RNA and RNA Free Water (RFW) were combined to make the volume of 12 ul, and 8 ul of miScript RT II Buffer (5× miScript Hiflex buffer 4 ul, 10× miScript Nucleics Mix 2 ul, miScript Reverse TranScriptase Mix 2 ul) was added to make the volume of 20 ul to carry out cDNA synthesis.
cDNA conditions were set as follows.

(When PrimeScript RT Master Mix was Used)
A. Stage 1: 37° C., 15 minutes
B. Stage 2: 85° C., 10° C. after 5 seconds, -ing

(When miScript RT II Kit was Used)
A. Stage 1: 37° C., 60 minutes
B. Stage 2: 95° C., 10° C. after 5 minutes, -ing

The synthesized cDNA was diluted by 1/20.

qRT-PCR was performed using primers having sequences described in Table 1. The primer used therefor was purchased from M.biotech (Hanam, Korea). By using 5 ul of qPCR Master Mix (2×, High ROX) (Gendepot, Cat #Q5602), qRT-PCR was performed in the total volume of 10 ul.

TABLE 1

| Gene | Accession No. | Forward sequence | Reverse sequence |
|---|---|---|---|
| SF3B4 | NM_005850.5 | SEQ ID NO: 1 | SEQ ID NO: 2 |
| HMBS | NM_001024382.2 | SEQ ID NO: 3 | SEQ ID NO: 4 |

The condition for qRT-PCR was set as follows.
A. Stage 1: 95° C., 2 minutes
B. Stage 2: 95° C., 15 seconds
C. 58° C., 34 seconds
D. 72° C., 30 seconds
E. →stage 2: 40 cycles.

4. Statistical Analysis of SF3B4 Results

After calculating relative concentration using the Microsoft Office Excel program, statistical analyses were performed using MedCalc statistical software and SPSS v22 analysis programs.

### <Example 1> Isolation of Exosomes for Identifying Specificity of SF3B4 as a Serum Exosomal Diagnostic Marker for Hepatocellular Carcinoma

In order to identify whether SF3B4 is expressed in exosomes, when SF3B4 expression in exosomes derived from blood of patients with various diseases was checked in the Exocarta database, it was found that SF3B4 was expressed in exosomes derived from blood of hepatocellular carcinoma patients.

Exosomes, which are a type of small extracellular vesicles produced in various cells, include various substances that show biological activity such as proteins, lipids, nucleic acids, and metabolites that cells have, and are known to exhibit functions as they are transferred to other cells. Since exosomes stably deliver substances, they are considered to be a major tool for clinical application in noninvasive liquid biopsy, and various research results have been reported, such that it was decided to measure expression of SF3B4 in RNA that was isolated from exosomes derived from sera, normal hepatic cell lines, and hepatocellular carcinoma cell lines.

In order to check that the serum exosomes were well isolated, nanoparticle tracking analysis (NTA) was performed to check the size of the isolated exosomes.

Next, when SF3B4 expression in cell lysates and exosomes of cell lines was identified, respectively, to find that SF3B4 is expressed in a higher level in hepatocellular carcinoma cell lines than normal hepatic cell lines, SF3B4 expression in hepatocellular carcinoma cell lines was higher in both cell lysates and exosomes than in normal hepatic cell lines.

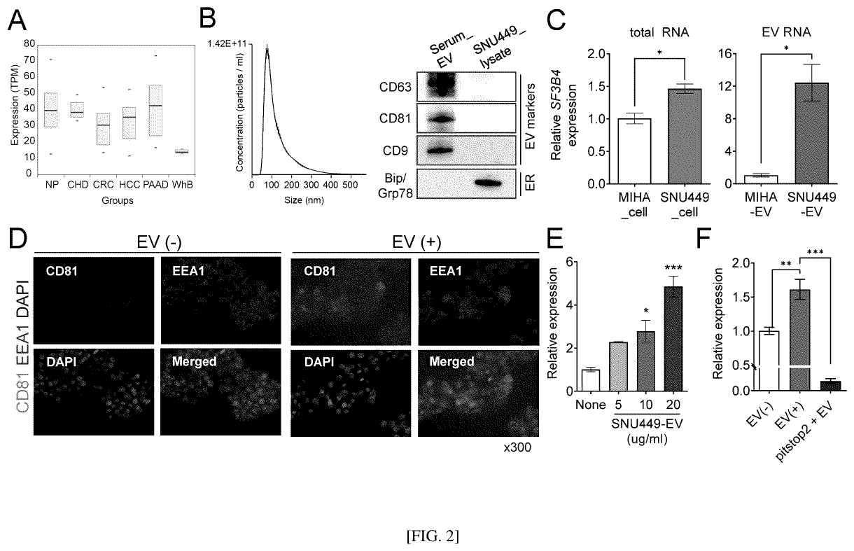

In addition, when exosomes of hepatocellular carcinoma cell lines were treated to normal hepatocytes to check that expression of SF3B4 increases by being delivered to other cells through exosomes, changes in SF3B4 expression were identified. It was found that when normal hepatocytes were treated with different concentrations of hepatocellular carcinoma cell-derived exosomes, SF3B4 expression increased. In addition, it was also found that when an endocytosis inhibitor was treated together, expression of SF3B4 did not increase even when exosomes were treated (FIG. 2).

### <Example 2> Evaluation on Diagnostic Performance of SF3B4 as a Serum Exosomal Diagnostic Marker for Hepatocellular Carcinoma

When expression was measured using qRT-PCR in buffy-coat and sera of 10 normal patients and 10 hepatocellular carcinoma patients to identify SF3B4 expression in the blood exosomes of hepatocellular carcinoma patients following the hepatocellular carcinoma cell lines, no difference was found in expression between normal patients and hepatocellular carcinoma patients. As a result of identifying expression of SF3B4 in extracted serum exosomal RNA, it was found that overexpression took place in all hepatocellular carcinoma patient samples, while expression barely took place in the normal group.

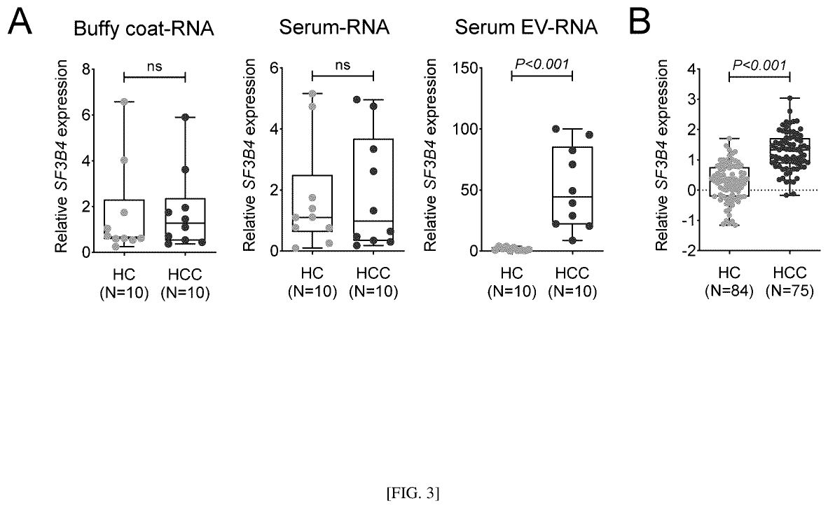

Furthermore, when SF3B4 expression was identified in serum exosomes of patients with and without hepatocellular carcinoma among patients having liver diseases, it was found that expression in hepatocellular carcinoma patients was higher than that in patients without hepatocellular carcinoma (FIG. 3).

### <Example 3> Evaluation on Diagnostic Performance of Exosomal SF3B4 in the Liver Disease Cohort

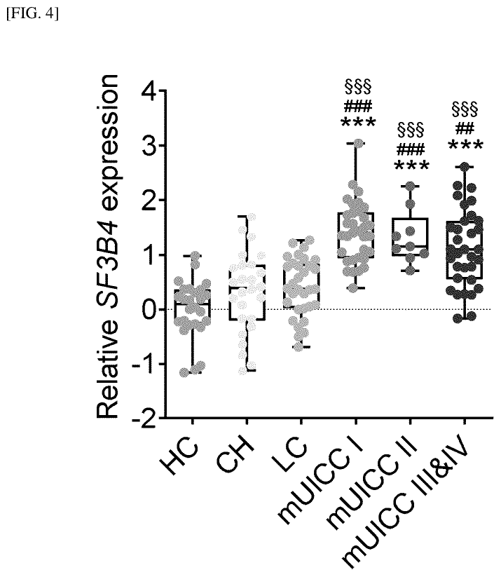

In order to evaluate the diagnostic performance of SF3B4 for hepatocellular carcinoma in patients visiting the Department of Gastroenterology at Ajou University Hospital, expression of SF3B4 was detected by qRT-PCR in serum exosomes of hepatocellular carcinoma patients with normal liver (n=26), chronic hepatitis (n=26), cirrhosis (n=32), mUICC I (n=33), mUICC II (n=9), and mUICC III/IV (n=33). Compared to non-hepatic cancer (HC, CH, LC), it was observed that SF3B4 expression in serum exosomes of hepatocellular carcinoma patients statistically, significantly increased (Welch's t-test, compare to normal liver; *P<0.05, **P<0.01, ***P<0.001, compare to CH; #P<0.05, ##P<0.01, ###P<0.001, compare to LC; § P<0.05, §§ P<0.01, §§§ P<0.001)(FIG. 4).

An expression cut-off value of exosomal SF3B4 in hepatocellular carcinoma patients over non-hepatic cancer (HC, CH, LC) was obtained by quantifying Ct values derived after performing qRT-PCR and converting a relative expression ratio to the normal group to log 10, resulting in the optimal point of 0.85. When SF3B4 was 0.85, the sensitivity was 76%, and the specificity was 85.19%.

### <Example 4> Results of Verification of Exosomal SF3B4 as a Marker for Early Stage Hepatocellular Carcinoma

In order to determine whether early stage hepatocellular carcinoma was detected, AUC was measured by dividing the mUICC stages, and then analysis was conducted by comparing with AFP, a conventional serum marker for hepatocellular carcinoma, so as to identify statistically significant superiority.

In hepatocellular carcinoma diagnosis over the entire hepatocellular carcinoma cohort, AUC of AFP was 0.673, and that of exosomal SF3B4 was 0.884, and, when compared with CH and LC which are high risk groups of hepatocellular carcinoma except the normal, AUC of AFP was 0.548 and that of exosomal SF3B4 was 0.854, showing significantly high diagnostic performance.

In hepatocellular carcinoma diagnosis in the early stage hepatocellular carcinoma cohort, including mUICC I and II, AUC of AFP was 0.582 and that of exosomal SF3B4 was 0.933, and, when compared with CH and LC which are high risk groups of hepatocellular carcinoma except the normal, AUC of AFP was 0.565 and that of exosomal SF3B4 was 0.910, showing a significant difference as well.

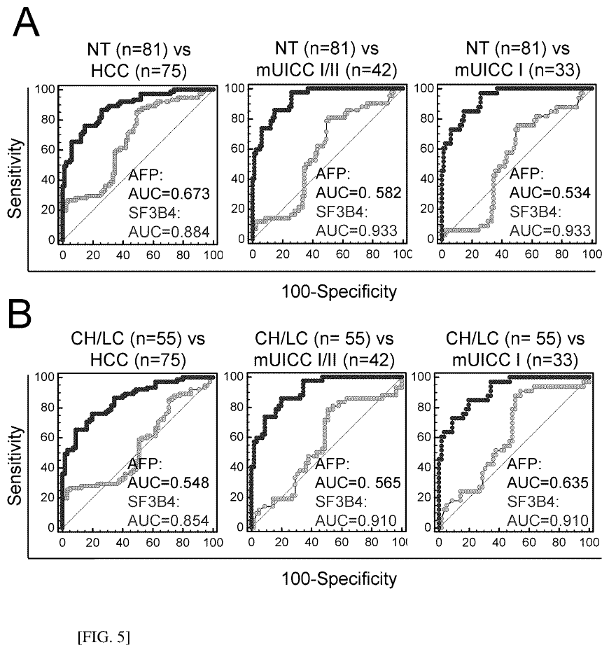

In the diagnosis of hepatocellular carcinoma in the early stage hepatocellular carcinoma cohort in the mUICC stage I with a tumor size less than or equal to 2 cm, AUC of AFP was 0.534, and that of exosomal SF3B4 was 0.933, and, when compared with CH and LC, which are high risk groups of hepatocellular carcinoma except the normal, AUC of AFP was 0.635 and that of exosomal SF3B4 was 0.910, showing a significant difference (FIG. 5).

Next, when the diagnostic performance was evaluated with a combination of AFP, a conventional blood marker, and serum exosomal SF3B4, AUC was 0.916 (95% CI: 0.861-0.954) in hepatocellular carcinoma diagnosis in the entire hepatocellular carcinoma cohort, and, when compared with CH and LC which are high risk groups of hepatocellular carcinoma except the normal, AUC was 0.894 (95% CI: 0.827-0.941), showing considerably high diagnostic performance.

In the diagnosis of hepatocellular carcinoma in the early stage hepatocellular carcinoma cohort including the mUICC I and II, AUC was 0.934 (95% CI: 0.875-0.971), and, when compared with CH and LC which are high risk groups of hepatocellular carcinoma except the normal, AUC was 0.910 (95% CI: 0.835-0.959), showing considerably high diagnostic performance.

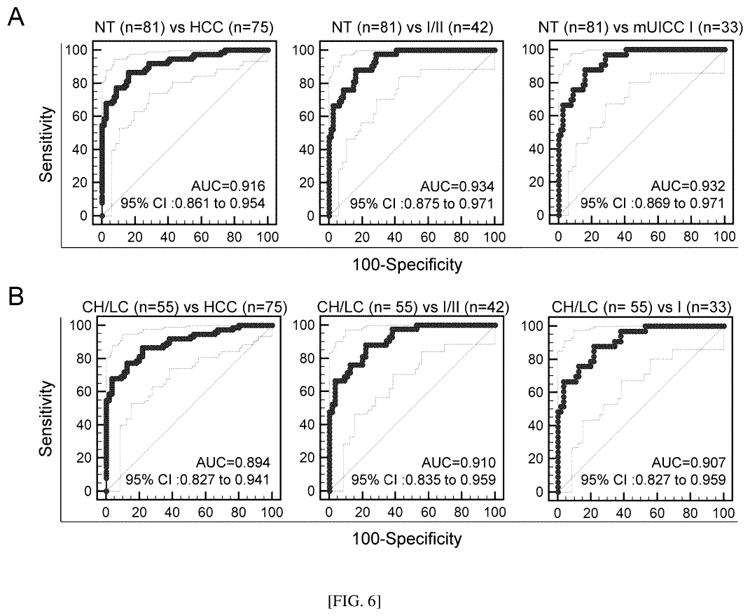

In the diagnosis of hepatocellular carcinoma in the early stage hepatocellular carcinoma cohort in the mUICC stage I, AUC was 0.932 (95% CI: 0.869-0.971), and, when compared with CH and LC which are high risk groups of hepatocellular carcinoma except the normal, AUC was 0.907 (95% CI: 0.827-0.959), showing considerably high diagnostic performance (FIG. 6).

Considering the positivity rate in normal liver, AFP was measured to be 0%, and exosomal SF3B4 to be 4%. In the hepatitis group, AFP was measured to be 35% and exosomal SF3B4 to be 18%. In the cirrhosis group, AFP was measured to be 56% and exosomal SF3B4 to be 22%. In the hepatocellular carcinoma patient group in the mUICC stage I, AFP was 12% and exosomal SF3B4 was 82%, measured in significantly higher positivity rates than AFP, a marker for hepatocellular carcinoma. In mUICC II, AFP was 56% and exosomal SF3B4 was 89%. In hepatocellular carcinoma patients in the mUICC III/IV, AFP was 59%, and exosomal SF3B4 was 64%. Overall, the positivity rate of exosomal SF3B4 was significantly lower than that of AFP in the non-hepatocellular carcinoma group, and the positivity rate of exosomal SF3B4 was significantly higher than that of AFP, especially in the early stage hepatocellular carcinoma group.

In comparison of the positivity rate of exosomal SF3B4 depending on the positivity of AFP in the entire group of hepatocellular carcinoma patients, the positivity of SF3B4 was 81% in patients with AFP-negative hepatocellular carcinoma.

In comparison of the positivity rate of SF3B4 depending on the positivity of AFP in the group of hepatocellular carcinoma patients in the UICC stage I/II, the positivity of exosomal SF3B4 was 85% in patients with AFP-negative hepatocellular carcinoma, showing very high diagnostic performance.

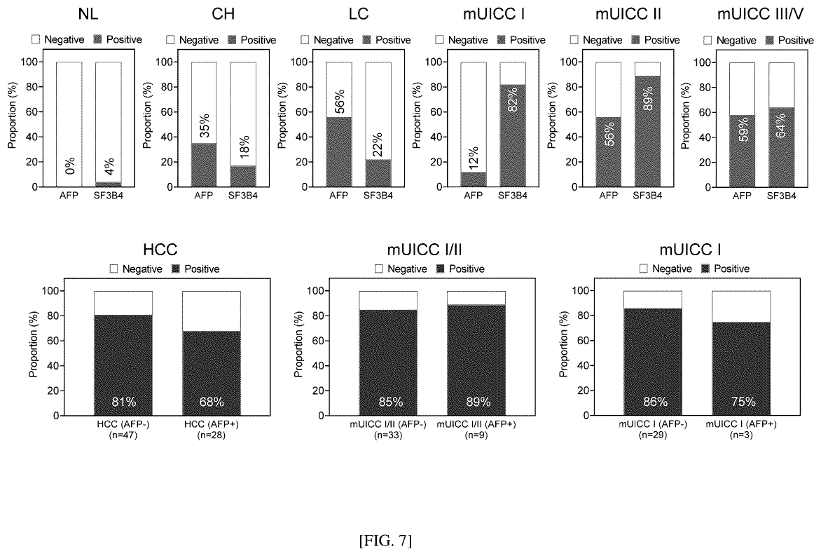

In comparison of the positivity rate of SF3B4 depending on the positivity of AFP in the group of hepatocellular carcinoma patients in the mUICC stage I, the positivity of exosomal SF3B4 was 86% in patients with AFP-negative hepatocellular carcinoma, showing potential as a marker that is capable of overcoming AFP with very low diagnostic performance for early stage hepatocellular carcinoma (FIG. 7).

### <Example 5> Diagnostic Performance of ELISA and autoAb SF3B4 Over Hepatocellular Carcinoma Other than Exosomal SF3B4

In addition to exosomes, additional techniques for diagnosing hepatocellular carcinoma were examined. As a result of ELISA using plasma of normal groups, hepatitis patients, cirrhosis patients, and hepatocellular carcinoma patients, SF3B4 expression in plasma of hepatocellular carcinoma patients was higher than in the normal group, but there was no significant difference in expression between non-hepatocellular carcinoma (normal, hepatitis, cirrhosis) patients and hepatocellular carcinoma patients. When an autoantibody against SF3B4 was measured, results were similar to those of ELISA.

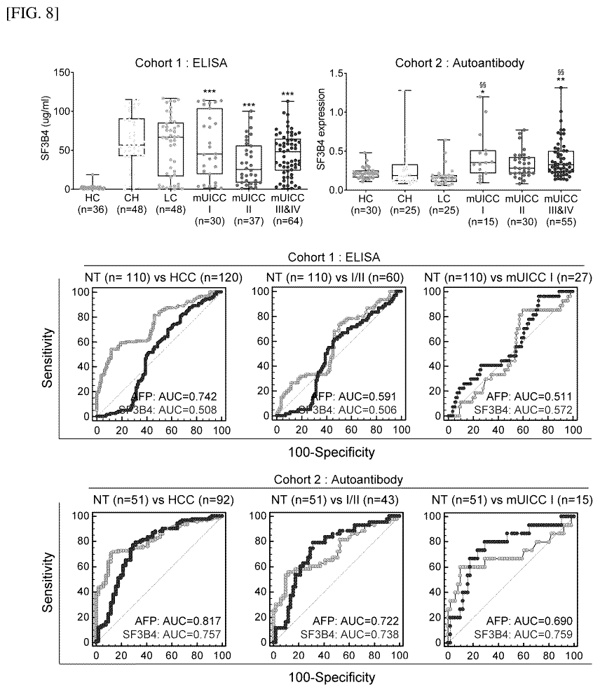

As a result of ROC analysis, it was found that an expression value of SF3B4 in both ELISA and autoantibody was not statistically, significantly higher than the diagnostic performance of AFP, which is conventionally used as a blood marker for hepatocellular carcinoma (FIG. 8).

As a specific part of the present disclosure is described in detail above, it will be apparent to those skilled in the art that such specific techniques are only preferred embodiments, and the scope of the present disclosure is not limited thereby. Accordingly, the substantial scope of the present disclosure will be defined by the appended claims and their equivalents.

What is claimed is:

1. A method of diagnosing hepatocellular carcinoma in a subject in need thereof, comprising:
providing a composition comprising a preparation capable of measuring an expression level of serum exosomal SF3B4 as an active ingredient; and
administering the composition to the subject, wherein the hepatocellular carcinoma is diagnosed.

2. The method of claim 1, wherein the preparation capable of measuring the expression level of the SF3B4 is a primer or probe that specifically binds to the SF3B4.

3. The method of claim 1, wherein the hepatocellular carcinoma is early stage hepatocellular carcinoma.

4. A method of providing information necessary for diagnosing hepatocellular carcinoma, comprising:
(1) measuring an expression level of SF3B4 from a blood-derived serum exosome sample isolated from a patient;
(2) comparing the measured expression level of SF3B4 with a control sample; and
(3) determining as hepatocellular carcinoma when the measured expression level of SF3B4 is higher than that of the control sample.

5. The method of claim 4, wherein the operation (3) is performed by a cut-off value which has a Ct value (threshold cycle) of 0.85 for the SF3B4.

6. The method of claim 4, wherein the hepatocellular carcinoma is early stage hepatocellular carcinoma.

7. A method of treating hepatocellular carcinoma, comprising administering an expression or activity inhibitor of serum exosomal SF3B4 to a hepatocellular carcinoma patient.

8. The method of claim 7, wherein the expression or activity inhibitor of SF3B4 is antisense nucleotides, small interfering RNAs (siRNAs), or short hairpin RNAs (shRNAs) that complementarily bind to mRNA of an SF3B4 gene, or compounds, peptides, peptide mimetics, aptamers, antibodies, or natural products that specifically bind to an SF3B4 protein.


SEQUENCE LISTING (4 sequences)

SEQ ID NO: 1 - DNA, 20 bp, synthetic construct
  source 1..20: mol_type = other DNA
agtcaacacccacatgccaa

SEQ ID NO: 2 - DNA, 21 bp, synthetic construct
  source 1..21: mol_type = other DNA
gggtccaggttcccaatgaaa

SEQ ID NO: 3 - DNA, 23 bp, synthetic construct
  source 1..23: mol_type = other DNA
ggagggcagaaggaagaaaacag

SEQ ID NO: 4 - DNA, 21 bp, synthetic construct
  source 1..21: mol_type = other DNA
cactgtccgtctgtatgcgag